(19) United States
(12) Patent Application Publication
(10) Pub. No.: US 2006/0211943 A1
(43) Pub. Date: Sep. 21, 2006

(54) Ultrasonic blade with terminal end balance features

(75) Inventor: Jean Beaupre, Cincinnati, OH (US)

Correspondence Address: JAMES C. EAVES JR.;GREENEBAUM DOLL & MCDONALD PLLC, 3500 NATIONAL CITY TOWER, 101 SOUTH FIFTH STREET, LOUISVILLE, KY, 40202, US

(73) Assignee: Crescendo Technologies, LLC

(21) Appl. No.: 11/348,911

(22) Filed: Feb. 7, 2006

Related U.S. Application Data
(60) Provisional application No. 60/661,738, filed on Mar. 15, 2005.

(51) Int. Cl. A61B 8/14

(52) U.S. Cl. 600/471

(57) ABSTRACT

The present invention is directed to methods and devices that provide balancing of an ultrasonic blade using terminal end balance features. An ultrasonic blade in accordance with embodiments of the present invention includes a terminal end non-functional balance feature in the functional portion of an asymmetric ultrasonic blade. Balancing in accordance with the present invention, using terminal end non-functional balance features, provides blade balance in a proximal portion of the blade, without the need for machining and alteration of blade shape in the functional portion of the blade, and without the reduction of mass and inherent stress increase proximal to the end-effector.

### FIELD OF THE INVENTION

The present invention relates, in general, to ultrasonic devices and, more particularly, to methods and devices that provide balancing of an ultrasonic blade using terminal end balance features.

### BACKGROUND OF THE INVENTION

The fields of ultrasonics and stress wave propagation encompass applications ranging from non-destructive testing in materials science, to beer packaging in high-volume manufacturing. Diagnostic ultrasound uses low-intensity energy in the 0.1-to-20-MHz region to determine pathological conditions or states by imaging. Therapeutic ultrasound produces a desired bio-effect, and can be divided further into two regimes, one in the region of 20 kHz to 200 kHz, sometimes called low-frequency ultrasound, and the other in the region from 0.2 to 10 MHz, where the wavelengths are relatively small, so focused ultrasound can be used for therapy. At high intensities of energy, this application is referred to as HIFU for High Intensity Focused Ultrasound.

Examples of therapeutic ultrasound applications are: HIFU for tumor ablation and lithotripsy, phacoemulsification, thrombolysis, liposuction, neural surgery and the use of ultrasonic scalpels for cutting and coagulation. In low-frequency ultrasound, direct contact of an ultrasonically active end-effector or surgical instrument delivers ultrasonic energy to tissue, creating bio-effects. Specifically, the instrument produces heat to coagulate and cut tissue, and cavitation to help dissect tissue planes. Other bio-effects include: ablation, accelerated bone healing and increased skin permeability for transdermal drug delivery.

At the tip of the end-effector, the energy is delivered to tissue to create several effects within the tissue. These include the basic gross conversion of mechanical energy to both frictional heat at the blade-tissue interface, and bulk heating due to viscoelastic losses within the tissue. In addition, there may be the ultrasonically induced mechanical mechanisms of: cavitation, microstreaming, jet formation and other mechanisms.

Ultrasonic medical devices are used for the safe and effective treatment of many medical conditions. Ultrasonic surgical instruments, and particularly solid core ultrasonic instruments, are advantageous because they may be used to cut and/or coagulate organic tissue using energy in the form of mechanical vibrations transmitted to a surgical end-effector at ultrasonic frequencies. Ultrasonic vibrations, when transmitted to organic tissue at suitable energy levels and using a suitable end-effector, may be used to cut, dissect, or cauterize tissue. Ultrasonic instruments utilizing solid core technology are particularly advantageous because of the amount of ultrasonic energy that may be transmitted from the ultrasonic transducer through the waveguide to the surgical end-effector. Such instruments are particularly suited for use in minimally invasive procedures, such as endoscopic or laparoscopic procedures, wherein the end-effector is passed through a trocar to reach the surgical site.

Ultrasonic vibration is induced in the surgical end-effector by, for example, electrically exciting a transducer which may be constructed of one or more piezoelectric or magnetostrictive elements in the instrument hand piece. Vibrations generated by the transducer section are transmitted to the surgical end-effector via an ultrasonic waveguide extending from the transducer section to the surgical end-effector. The waveguides and end-effectors are designed to resonate at the same frequency as the transducer. Therefore, when an end-effector is attached to a transducer the overall system frequency is still the same frequency as the transducer itself.

Solid core ultrasonic surgical instruments may be divided into two types, single element end-effector devices and multiple-element end-effector. Single element end-effector devices include instruments such as scalpels, and ball coagulators, see, for example, U.S. Pat. No. 5,263,957. Multiple element end-effectors include those illustrated in devices such as ultrasonic shears, for example, those disclosed in U.S. Pat. Nos. 5,322,055 and 5,893,835 provide an improved ultrasonic surgical instrument for cutting/coagulating tissue, particularly loose and unsupported tissue. The ultrasonic blade in a multiple-element end-effector is employed in conjunction with a clamp for applying a compressive or biasing force to the tissue. Clamping the tissue against the blade provides faster and better controlled coagulation and cutting of the tissue.

In an ultrasonic device running at resonance in primarily a longitudinal mode, the longitudinal ultrasonic motion, d, behaves as a simple sinusoid at the resonant frequency as given by:
d=A sin(ω·t)
where: ω=the radian frequency, which equals (2·π) multiplied by the cyclic frequency, f; t is time; and A=the zero-to-peak amplitude.

The longitudinal excursion is defined as the peak-to-peak amplitude, which is twice the amplitude of the sine wave, mathematically expressed as 2·A.

A blade in perfect balance over its entire length will vibrate longitudinally according to this simple harmonic motion. Unfortunately, ultrasonic blades are not typically in perfect balance. For example, blades useful for medical applications may incorporate curves or features that cause blade imbalances.

### SUMMARY OF THE INVENTION

The present invention is directed to methods and devices that provide balancing of an ultrasonic blade using terminal end balance features. An ultrasonic blade in accordance with embodiments of the present invention includes a terminal end nonfunctional balance feature in the functional portion of an asymmetric ultrasonic blade. Balancing in accordance with embodiments of the present invention, using terminal end non-functional balance features, provides blade balance in a proximal portion of the blade, without the need for machining and alteration of blade shape in the functional portion of the blade, and without the reduction of mass and inherent stress increase proximal to the end-effector.

The above summary of the present invention is not intended to describe each embodiment or every implementation of the present invention. Advantages and attainments, together with a more complete understanding of the invention, will become apparent and appreciated by referring to the following detailed description and claims taken in conjunction with the accompanying drawings.

### BRIEF DESCRIPTION OF THE DRAWINGS

The features of the invention may be set forth with particularity in the appended claims. The invention itself, however, both as to organization and methods of operation, together with further objects and advantages thereof, may best be understood by reference to the following description, taken in conjunction with the accompanying drawings in which:

FIG. 1 is a pictorial view of an ultrasonic blade having terminal end balance features in accordance with an embodiment of the present invention;

FIG. 2 is a side view of the ultrasonic blade having terminal end balance features in accordance with embodiments of the present invention as illustrated in FIG. 1;

FIG. 3 is a pictorial view of an ultrasonic blade having terminal end balance features in accordance with another embodiment of the present invention;

FIG. 4 is a side view of the ultrasonic blade having terminal end balance features in accordance with embodiments of the present invention as illustrated in FIG. 3;

FIG. 5a is a side view of an ultrasonic blade having a functional asymmetry, wherein the blade is not balanced;

FIG. 5b is a side view of an ultrasonic blade having a functional asymmetry, wherein the blade is balanced in accordance with embodiments of the present invention;

FIG. 5c is a magnified side view of the proximal portion of the ultrasonic blade illustrated in FIG. 5a, illustrating the non-longitudinal motion of the blade imbalance;

FIG. 5d is a magnified side view of the proximal portion of the ultrasonic blade illustrated in FIG. 5b, illustrating the balanced longitudinal motion of the blade;

FIG. 6a is a side view of the ultrasonic waveguide portion of the blade illustrated in FIG. 5a; and

FIG. 6b is a side view of the ultrasonic waveguide portion of the blade illustrated in FIG. 5b.

While the invention is amenable to various modifications and alternative forms, specifics thereof have been shown by way of example in the drawings and will be described in detail below. It is to be understood, however, that the intention is not to limit the invention to the particular embodiments described. On the contrary, the invention is intended to cover all modifications, equivalents, and alternatives falling within the scope of the invention as defined by the appended claims.

### DETAILED DESCRIPTION OF THE INVENTION

In the following description of the illustrated embodiments, references are made to the accompanying drawings, which form a part hereof, and in which is shown by way of illustration various embodiments in which the invention may be practiced. It is to be understood that other embodiments may be utilized, and structural and functional changes may be made without departing from the scope of the present invention.

Considerable effort has been directed at correcting imbalances inherent in curved ultrasonic blades and ultrasonic devices that are not symmetric about their longitudinal axis. Descriptions of methods to correct ultrasonic blade imbalances are described in U.S. Pat. Nos. 6,283,981; 6,328,751; 6,660,017; 6,325,811; 6,432,118; and 6,773,444, the content of which are hereby incorporated herein by reference. Although balancing of ultrasonic blades has greatly expanded the possibilities of blade design, balancing using the methodologies described in U.S. Pat. Nos. 6,283,981; 6,328,751; and 6,660,017 require balance asymmetries proximal to the functional portion of the blade. Balancing methodologies described in U.S. Pat. Nos. 6,325,811; 6,432,118; and 6,773,444 describe the use of functional asymmetries in the end-effector that may be used for balancing.

Balancing using asymmetries proximal to the end-effector using reductions of mass inherently causes reduction in strength due to the lost mass at the balance asymmetry. Balancing using asymmetries in the end-effector, such as is described in U.S. Pat. Nos. 6,325,811; 6,432,118; and 6,773,444 require machining and alteration of blade shape in the functional portion of the blade. Balancing in accordance with the present invention, using terminal end non-functional balance features, provides blade balance in a proximal portion of the blade, without the need for machining and alteration of blade shape in the functional portion of the blade, and without the reduction of mass and inherent stress increase proximal to the end-effector. It is understood that a non-functional balance feature is feature that functions to balance the ultrasonic blade, but may or may not serve a clinical function, e.g. the balance feature may or may not come into contact with tissue.

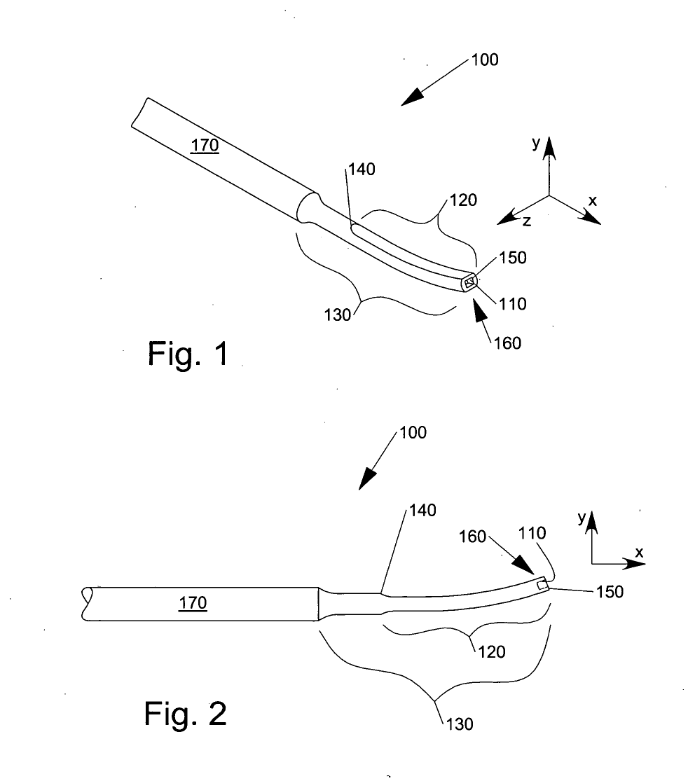

Referring now to FIG. 1, a pictorial view of an ultrasonic blade 100 is illustrated having a terminal end balance feature 110 in a functional portion 120 of an end-effector 130. The functional portion 120 is illustrated as beginning at a first point 140 and terminating at a terminal point 150. The terminal end balance feature 110 extends into the functional portion 120 from the terminal point 150. The ultrasonic blade 100 includes, in this example, the end-effector 130 distal to a waveguide 170.

The terminal end balance feature 110, illustrated in this embodiment as a hollow portion 160, may be created, for example, by drilling into the functional portion 120 from the terminal point 150. The terminal end balance feature 110 may be drilled in to a depth that balances the non-longitudinal motion proximal to the end-effector 130 created by the asymmetry due to the functional portion 120, in this example, having a curvature in the y-direction. The amount of material needed to be removed, thereby creating the terminal end balance feature 110, may be determined analytically. For example, the methodologies described in U.S. Pat. No. 6,325,811 previously incorporated by reference, may be used to calculate the amount of mass that must be removed to offset a given functional asymmetry. The mass that needs to be removed to balance an asymmetry may also be determined empirically by incrementally drilling into the end-effector 130 and measuring the non-longitudinal motion proximal to the end-effector 130, such as by using a laser vibrometer or other method known in the art. Drilling to increasing depths may be iterated with measurements until a balance point is found.

The ultrasonic blade 100 is preferably made from a solid core shaft constructed of material which propagates ultrasonic energy, such as a titanium alloy (i.e., Ti-6AI-4V) or an aluminum alloy. It will be recognized that the ultrasonic blade 100 may be fabricated from any other suitable material. It is also contemplated that the ultrasonic blade 100 may have a surface treatment to improve the delivery of energy and desired tissue effect. For example, the ultrasonic blade 100 may be micro-finished, coated, plated, etched, grit-blasted, roughened or scored to enhance coagulation and cutting of tissue and/or reduce adherence of tissue and blood to the end-effector 130. Additionally, the ultrasonic blade 100 may be sharpened or shaped to enhance its characteristics. For example, the functional portion 120 of the ultrasonic blade 100 may be blade shaped, hook shaped, ball shaped, a straight right-circular cylinder, a curved right-circular cylinder, or other desired shape.

FIG. 2 is a side view of the ultrasonic blade 100, with the terminal end balance feature 110 in the functional portion 120 of the end-effector 130 as is illustrated in FIG. 1. The terminal end balance feature 110 depth into the functional portion 120 from the terminal point 150 illustrated in FIG. 2 is sufficient to balance the ultrasonic blade 100. Stresses in the ultrasonic blade 100 at the terminal end anti-node approach zero in a non-loaded blade. The loss of material due to the terminal end balance feature 110 has a negligible effect on the strength and/or functionality of the end-effector 130. In contrast to the functional balance asymmetries illustrated in U.S. Pat. No. 6,325,811, for example, the functional area of the ultrasonic blade 100 may have a constant cross-section outer surface.

An ultrasonic end-effector 130 with an ultrasonic blade 100 that has multiple asymmetries will naturally have a tendency to include tip excursion in at least two, and possibly all three axes, x, y, and z. If not balanced properly, excursions other than longitudinal will reflect a moment or force back to the transducer, causing inefficiencies and/or loss of lock to the longitudinal drive frequency, and possibly blade fracture. For example, ultrasonic blade 100 as illustrated in FIGS. 1 and 2 is curved in the y direction at its distal end. This curvature will cause ultrasonic blade 100 to have excursions in at least both the x and y directions when activated.

It is possible to balance forces and/or moments caused by non-longitudinal tip excursion of the functional asymmetry using terminal end balance features in accordance with the present invention. It is desirable to balance ultrasonic blade 100 below 15% non-longitudinal excursion proximal to the functional asymmetry and it is preferable to balance ultrasonic blade 100 below 5% non-longitudinal excursion proximal to the functional asymmetry.

A normalized non-longitudinal excursion percentage in an ultrasonic blade may be calculated by taking the magnitude of the excursion in the non-longitudinal direction, and dividing that magnitude by the magnitude of the maximum vibration excursion in the longitudinal direction (also called the primary vibration excursion), and then multiplying the dividend by one hundred. Primary tip vibration excursion is the magnitude of the major axis of the ellipse or ellipsoid created by a point on the distal most end, designated the terminal end, of ultrasonic blade 100 when the ultrasonic blade 100 is activated.

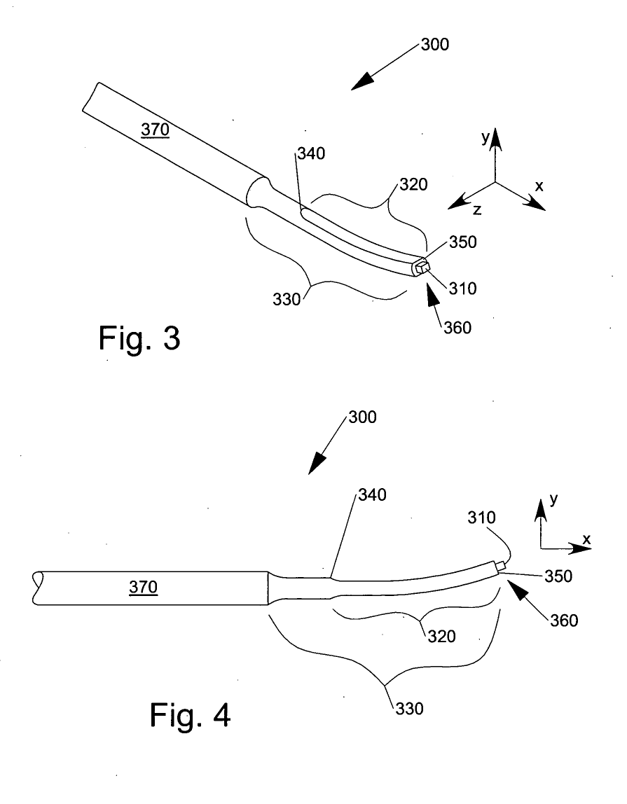

FIG. 3 is a pictorial view of an ultrasonic blade 300 having terminal end balance features in accordance with another embodiment of the present invention. The blade 300 includes a terminal end balance feature 310 in a functional portion 320 of an end-effector 330. The functional portion 320 is illustrated as beginning at a first point 340 and terminating at a terminal point 350. The terminal end balance feature 310 extends from the terminal point 350. The ultrasonic blade 300 includes, in this example, the end-effector 330 distal to a waveguide 370.

The terminal end balance feature 310, illustrated in this embodiment as a solid extension 360, may be created, for example, during the machining of the blade 300. The terminal end balance feature 310 may extend to a length that balances the non-longitudinal motion proximal to the end-effector 330 created by the asymmetry due to the functional portion 320, in this example, having a curvature in the y-direction.

FIG. 4 is a side view of the ultrasonic blade 300 having the terminal end balance feature 310 in accordance with embodiments of the present invention as illustrated in FIG. 3. The terminal end balance feature 310 extends from the terminal point 150 illustrated in FIG. 2 a sufficient length to balance the ultrasonic blade 100. The terminal end balance feature 310 may be useful if additional mass is needed to balance the blade 300 at the proximal waveguide portion 370, instead of reducing the mass at the terminal end as is illustrated in FIGS. 1 and 2.

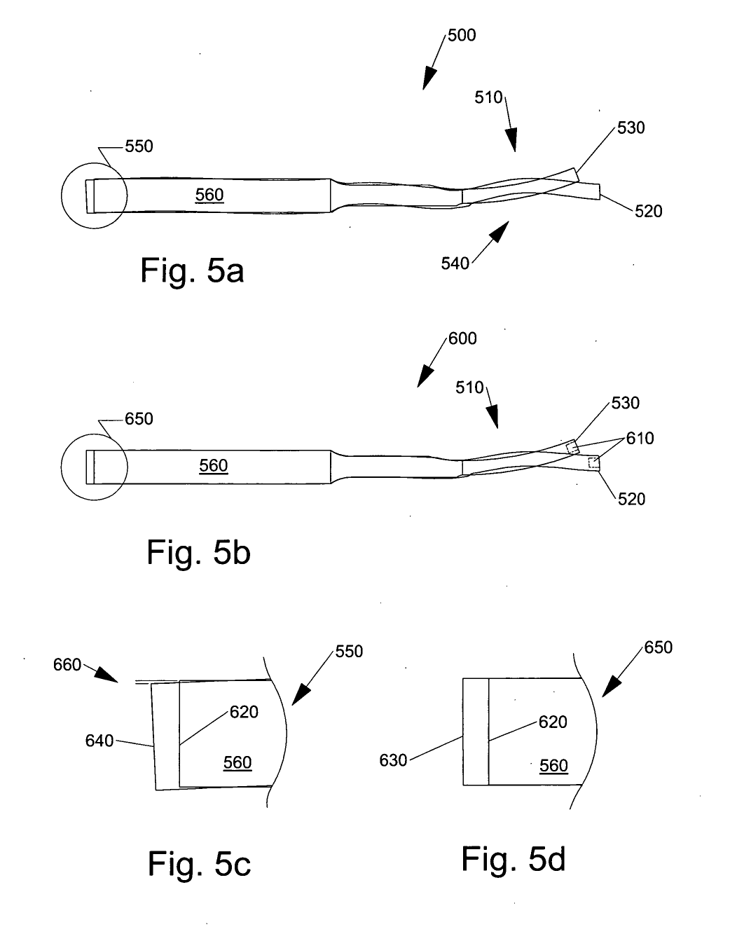

FIG. 5a is a side view of an ultrasonic blade 500 having a functional asymmetry 510, wherein the blade is not balanced. The blade 500 is illustrated at a maximum excursion 520 and a zero excursion 530 of a functional portion 540. A proximal portion 550 of a waveguide 560 section of the blade 500 will be described in more detail when referring to FIG. 5c below.

FIG. 5b is a side view of a balanced version 600 of the ultrasonic blade 500 Illustrated in FIG. 5a. The balanced version 600 is balanced in accordance with embodiments of the present invention using a terminal end balance feature 610, similar to the feature illustrated in FIGS. 1 and 2. Otherwise, the balanced version 600 has other features similar to the ultrasonic blade 500. A proximal portion 650 of a waveguide 560 section of the balanced version 600 will be described in more detail when referring to FIG. 5d below.

FIGS. 5c and 5d may be compared to illustrate the differences between the motions produced in the ultrasonic blade 500 versus the balanced version 600 from excitation by a pure longitudinal driving motion. FIG. 5c is a magnified side view of the proximal portion of the ultrasonic blade 500 illustrated in FIG. 5a, illustrating the non-longitudinal motion of the blade imbalance. FIG. 5d is a magnified side view of the proximal portion of the balanced version 600 illustrated in FIG. 5b, illustrating the balanced longitudinal motion of the blade.

In FIGS. 5c and 5d, a proximal face 620 of the waveguide portion 560 is illustrated, corresponding to a location of the proximal face 620 at a zero crossing of the longitudinal excursion. FIG. 5d illustrates a face position 630, corresponding to the proximal-most excursion of the proximal face 620 during ultrasonic motion. The proximal face 620 and the face position 630 are illustrated to be parallel, indicating pure longitudinal motion of the waveguide 560 section, due to balance provided by the terminal end balance feature 610.

FIG. 5c illustrates a face position 640, also corresponding to the proximal-most excursion of the proximal face 620 during ultrasonic motion. The proximal face 640 and the face position 630 are illustrated not parallel to each other, and also include a lateral motion offset 660. The offset 660 indicates non-longitudinal motion of the waveguide 560 section, due to imbalance caused by the functional asymmetry 510. Furthermore, the non-parallel aspect of the face position 640 indicates that the transducer driving the ultrasonic blade 500 imbalanced will have decreased efficiency, increased wear, and possibly overheating and failure.

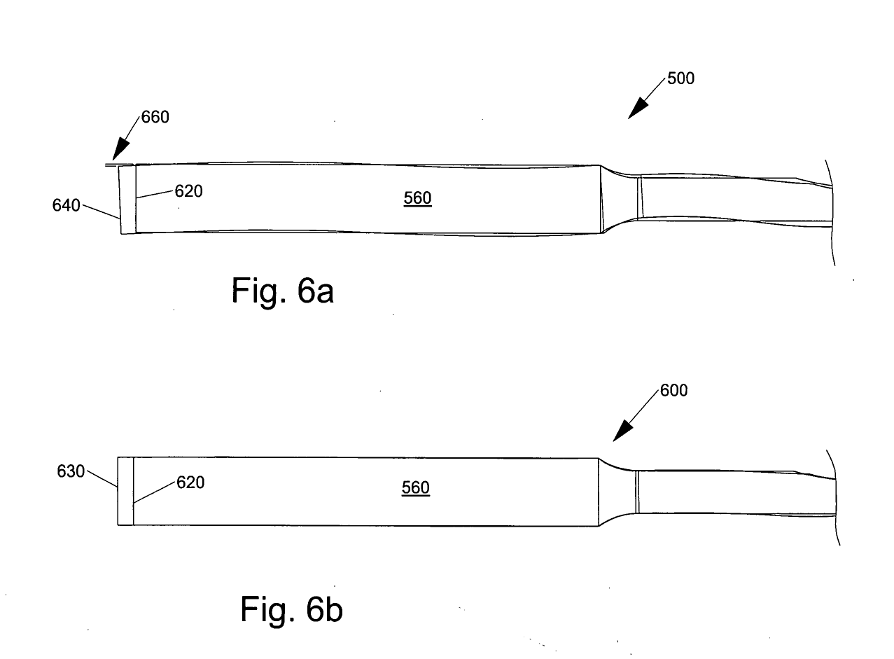

FIG. 6a is a side view of the ultrasonic waveguide portion 560 of the blade 500 illustrated in FIG. 5a, and FIG. 6b is a side view of the ultrasonic waveguide portion 560 of the balanced version 600 illustrated in FIG. 5b. In addition to the proximal face 620 and the lateral motion offset 660 seen in FIG. 5c, FIG. 6A illustrates that the lateral motion offset 660 corresponds to non-longitudinal motion throughout the blade. Whereas FIG. 6b illustrates that the waveguide 560 section proximal to the functional asymmetry 510 is in balance for the balanced version 600 that uses a terminal end balance feature in accordance with the present invention.

Each feature disclosed in this specification (including any accompanying claims, abstract, and drawings), may be replaced by alternative features having the same, equivalent or similar purpose, unless expressly stated otherwise. Thus, unless expressly stated otherwise, each feature disclosed is one example only of a generic series of equivalent or similar features.

While embodiments of the present invention have been shown and described herein, it will be obvious to those skilled in the art that such embodiments are provided as examples only. Numerous variations, changes, and substitutions will be apparent to those skilled in the art without departing from the invention. Accordingly, it is intended that the invention be limited only by the scope of the appended claims.

What is claimed is:

1. An ultrasonic blade, comprising:
a waveguide configured to transmit ultrasonic energy therethrough;
an end-effector provided at the distal end of the waveguide, the end-effector comprising a functional asymmetry; and
a means for balancing the non-longitudinal excursion in the waveguide proximal to the end-effector, the balancing means comprising a terminal-end balance feature.

2. An ultrasonic blade according to claim 1 wherein the balancing means comprises a non-functional balance feature.

3. An ultrasonic blade according to claim 1 wherein the non-longitudinal motion in the waveguide is less than 15% of the longitudinal motion.

4. An ultrasonic blade according to claim 1 wherein the non-longitudinal motion in the waveguide is less than 5% of the longitudinal motion.

5. An ultrasonic blade according to claim 1 wherein the functional asymmetry is curved.

6. An ultrasonic blade according to claim 1 wherein the functional asymmetry is not surgically sharp.

7. An ultrasonic blade according to claim 1 wherein the ultrasonic blade further comprises a means for compressing tissue.

8. An ultrasonic instrument comprising:
an ultrasonic waveguide having a distal end,
an end-effector located at the distal end of said ultrasonic waveguide,
said end-effector comprising a functional asymmetry,
said functional asymmetry comprising an energy-conductive portion,
a means for balancing non-longitudinal excursion in the ultrasonic waveguide proximal to the end-effector due to said functional asymmetry, said balancing means comprising a terminal-end balance feature.

9. An ultrasonic instrument according to claim 8 wherein the balance feature is non-functional.

10. An ultrasonic instrument according to claim 8 wherein the non-longitudinal motion in the ultrasonic waveguide is less than 15% of the longitudinal motion.

11. An ultrasonic instrument according to claim 8 wherein the non-longitudinal motion in the ultrasonic waveguide is less than 5% of the longitudinal motion.

12. An ultrasonic instrument according to claim 8 wherein the functional asymmetry is curved.

13. An ultrasonic instrument according to claim 8 wherein the functional asymmetry is not surgically sharp.

14. An ultrasonic instrument according to claim 8 wherein the ultrasonic instrument further comprises a means for compressing tissue.

15. A method of balancing non-longitudinal excursion in an ultrasonic waveguide of an ultrasonic instrument, comprising:
providing an ultrasonic instrument having an ultrasonic waveguide with a distal end;
providing an end-effector having a functional asymmetry at the distal end of the waveguide; and
balancing non-longitudinal excursion in the ultrasonic waveguide by selectively shaping a terminal end of the end-effector to provide a terminal end depression or extension.
